(19) United States
(12) Patent Application Publication
(10) Pub. No.: US 2016/0334643 A1
(43) Pub. Date: Nov. 17, 2016

(54) CHROMATIC ABERRATION CORRECTION LENS SYSTEMS AND METHODS

(71) Applicant: Elwha LLC, Bellevue, WA (US)

(72) Inventors: Roderick A. Hyde, Redmond, WA (US); Yaroslav A. Urzhumov, Bellevue, WA (US); Lowell L. Wood,, JR., Bellevue, WA (US)

(73) Assignee: Elwha LLC, Bellevue, WA (US)

(21) Appl. No.: 14/713,844

(22) Filed: May 15, 2015

(51) Int. Cl. G02C 7/08; A61F 2/16; G02C 7/04; G02C 7/02; G02C 7/06

(52) U.S. Cl. CPC G02C 7/086; G02C 7/022; G02C 7/06; G02C 7/04; A61F 2/1648; A61F 2/1637; G02C 2202/20; G02C 2202/22; G02C 2202/16

(57) ABSTRACT

A wearable lens assembly includes a first lens having a first optical power; and a second lens coupled to the first lens and having a second optical power substantially equal in magnitude and opposite in power to the first optical power. The first and second lenses are collectively configured to correct chromatic aberration in at least one of an eye of a user and corrective eyewear of a user.

### BACKGROUND

The human eye receives light rays generated by various light sources (the sun, etc.) and light rays reflected off of various objects. The light rays enter the eye through the cornea and pass through a lens that focuses the light rays at the retina at the back of the eye. Optical materials such as the cornea and the lens in the eye have different refractive indices for different wavelengths (e.g., colors) of light. As such, due to the inherent characteristics of the lens, different colors tend to focus at different convergent points, resulting in chromatic aberration seen as fringes of color appearing, for example, along boundaries separating dark and light objects. Should a person use corrective eyewear, the lenses of such eyewear may introduce additional chromatic aberration.

### SUMMARY

One embodiment relates to a wearable lens assembly, including a first lens having a first optical power; and a second lens coupled to the first lens and having a second optical power substantially equal in magnitude and opposite in power to the first optical power; wherein the first and second lenses are collectively configured to correct chromatic aberration in at least one of an eye of a user and corrective eyewear of a user.

Another embodiment relates to a supplemental eyeglass assembly configured to supplement primary corrective eyewear, including a first lens assembly configured to correct a first chromatic aberration resulting from a first eye of a user and a first primary lens of the primary corrective eyewear; and a second lens assembly configured to correct a second chromatic aberration resulting from a second eye of the user and a second primary lens of the primary corrective eyewear; wherein the first and second lens assemblies are configured to be coupled to the primary corrective eyewear and have substantially no effect on the achromatic focusing power of the primary corrective eyewear.

Another embodiment relates to a corrective eyeglass assembly, comprising a primary wearable lens assembly configured to provide a focal shift to light incident upon a user eye at a reference wavelength; and a secondary wearable lens assembly separable from the primary wearable lens assembly and configured to correct a chromatic aberration resulting from at least one of the user eye and the primary wearable lens assembly; wherein the secondary wearable lens assembly has substantially no effect on the focal shift provided by the primary wearable lens assembly at the reference wavelength.

Another embodiment relates to a method of making a lens assembly, including providing a user with a plurality of lens samples, each lens sample providing a different amount of chromatic correction; receiving an input from the user regarding a selection of one of the plurality of lens samples; and providing the use with a lens assembly based on the selection, the lens assembly configured to correct a degree of chromatic aberration.

Another embodiment relates to a method of applying a supplemental lens assembly to a primary lens, including receiving a user input regarding a degree of chromatic aberration resulting from a primary lens and a user eye; determining a profile of the primary lens; selecting a supplemental lens assembly template based on the degree of chromatic aberration; modifying the supplemental lens assembly template based on the profile of the primary lens and the degree of chromatic aberration to form the supplemental lens assembly; and coupling the supplemental lens assembly to the primary lens.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a perspective view of a human eye receiving light rays according to one embodiment.

FIG. 2 is a cross-section view of a human eye according to one embodiment.

FIG. 3 is a schematic illustration of a lens of an eye and chromatic aberrations according to one embodiment.

FIG. 4 is a schematic illustration of chromatic aberration resulting from an eye lens and corrective eyewear.

FIG. 5 is a schematic illustration of a supplemental lens assembly usable with the eye lens and corrective eyewear of FIG. 4 according to one embodiment.

FIG. 6 is an illustration of a supplemental lens assembly according to one embodiment.

FIG. 7 is an illustration of a diffractive lens profile according to one embodiment.

FIG. 8 is an illustration of a diffractive lens profile according to another embodiment.

FIG. 9 is a perspective view of an eye diagnostic system according to one embodiment.

FIGS. 10-12 illustrate various degrees of chromatic aberration according to various embodiments.

FIGS. 13-15 illustrate various supplemental lens assemblies according to various embodiments.

FIG. 16 is a schematic block diagram of an eye evaluation and lens fabrication system according to one embodiment.

FIG. 17 is a schematic illustration of an eye evaluation and supplemental lens fabrication system according to one embodiment.

FIG. 18 is a block diagram illustrating a method of evaluating an eye and fabricating a supplemental lens according to one embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring to the figures generally, various embodiments disclosed herein relate to correcting occurrences of optical aberrations existing within the human eye and/or introduced by corrective eyewear (e.g., glasses, contact lenses, etc.), and more specifically, to correcting chromatic aberrations existing within the eye or introduced by corrective eyewear. The lens of the human eye is a biconvex lens, and has different refractive indices for different wavelengths (e.g., colors) of light. As such, due to the inherent characteristics of the lens, different colors tend to focus at different convergent points, resulting in fringes of color appearing, for example, along boundaries separating dark and light objects. Correction of chromatic aberrations may include, for example, making various wavelengths (e.g., colors) or light focus closer to a common convergence point. Such correction may or may not further include adjusting the overall (achromatic) focusing power of the eye (or other primary lens).

As with the majority of optical materials, the refractive indices for materials in the eye are larger for short wavelength light (e.g., blue light) than for long wavelength light (e.g., red light). Accordingly, blue light focuses at a shorter distance within the eye than red light does. As a consequence, if one color is focused on the retina, other colors will not be. For example, if green light is focused on the retina, then red light will be focused slightly behind it, while blue light will be focused above the retina. Hence, multi-color objects are inherently somewhat blurry, and due to the nonlinearity of the dependence of refractive index with wavelength, this blurriness is generally more pronounced for blue than for other colors. Lenses of corrective eyewear suffer from similar chromatic aberration issues as those existing within the eye, due to the inherent characteristics of the optical materials used for corrective eyewear lenses.

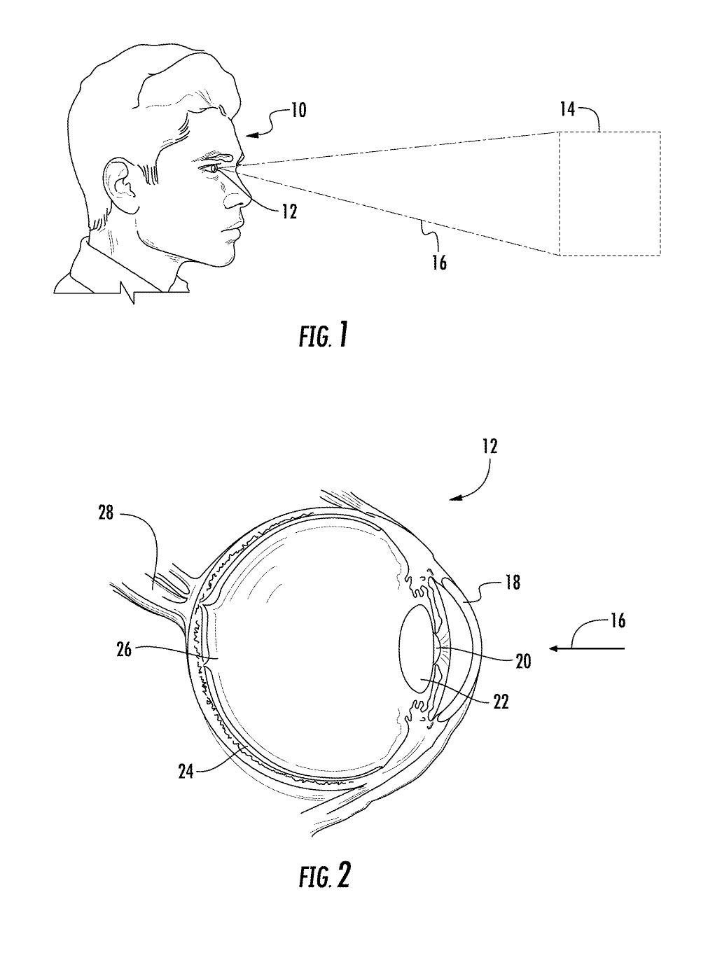

Referring now to FIGS. 1-2, eye 12 of person 10 is shown according to one embodiment. Eye 12 receives light 16 (e.g., light rays or electromagnetic radiation having wavelengths within the visible spectrum) from various sources, including sunlight, light reflected by objects such as object 14, etc. Light 16 enters eye 12 through cornea 18. Cornea 18 is a substantially transparent outer covering of the eye that bends or refracts the light. Light rays are refracted, or bent, due to differences in the refractive index of the light during its transmission, e.g., across surfaces between two differing materials, such as the front and back surfaces of cornea 18 and lens 22. From the cornea, the light passes through pupil 20 and lens 22. Lens 22 further bends or refracts light 16 such that light 16 (ideally) is focused on retina 24 at the back of the eye. Retina 24 is a relatively thin layer of tissue at the back of the eye, and includes light-sensing nerve cells concentrated in macula 26. The nerve cells convert the sensed light to electrical impulses, which are transported to the brain via optical nerve 28.

Ideally, as light passes through lens 22, the various light rays (e.g., corresponding to the different wavelengths/colors of light) focus on a single focal point. However, as noted above, lenses inherently refract light rays of different wavelengths differently. As such, rather than converging on a single convergent focal point, light rays of different wavelengths or colors tend to converge at various different points along the optical axis of the lens. This results in optical distortion referred to as chromatic aberration. The focal point can be displaced longitudinally (i.e., longitudinal chromatic aberration) or laterally (i.e., lateral chromatic aberration). Other optical aberrations causing imperfect focusing occur within the eye, including astigmatism, coma, spherical aberration, and others. Because the refractive indices are wavelength dependent, such optical aberrations are color dependent as well. For conceptual purposes, an optical power or an aberration may be split into the sum of two parts, one (the achromatic part) which is independent of wavelength, and a second (the chromatic part) which encompasses the wavelength dependence. The achromatic part can be chosen to be the value of the optical power or aberration at a reference wavelength (e.g., for green light). Alternatively, the achromatic part can be chosen to be the average value of the optical power or aberration over a specified wavelength region (e.g., for visible light). Other reference wavelength(s) for defining the achromatic part of an optical power or aberration may be selected in other embodiments. The chromatic portion of the optical power or aberration can be expressed as the full spectrally dependent value minus the achromatic value. This apportionment can be mathematically represented below, splitting a fully spectrally dependent optical power or aberration Φ(λ) into an achromatic part Φ(λ) denoting the value at a reference wavelength λ* and a chromatic part Ψ(λ−λ*) functionally depending on the difference between a wavelength and the reference wavelength:

Φ(λ)≡Φ(λ*)+Ψ(λ−λ*)

In general the chromatic portion is a nonlinear function of wavelength. However, for limited spectral ranges near the reference wavelength, the dominant chromatic variation is linear with wavelength, allowing a simpler formulation:

Φ(u)≈Φ*[1−αu] where u≡λ/λ*−1

where u is a dimensionless spectral variable representing the change in wavelength, Φ* represents the achromatic value of the optical power or aberration, and α is a dimensionless constant denoting the relative size of the chromatic part to that of the achromatic part (generally α is relatively small, e.g., ˜0.1 or less). The use of subtraction rather than addition reflects the fact that index of refraction based optical properties generally weaken at longer wavelengths (i.e., at positive u values).

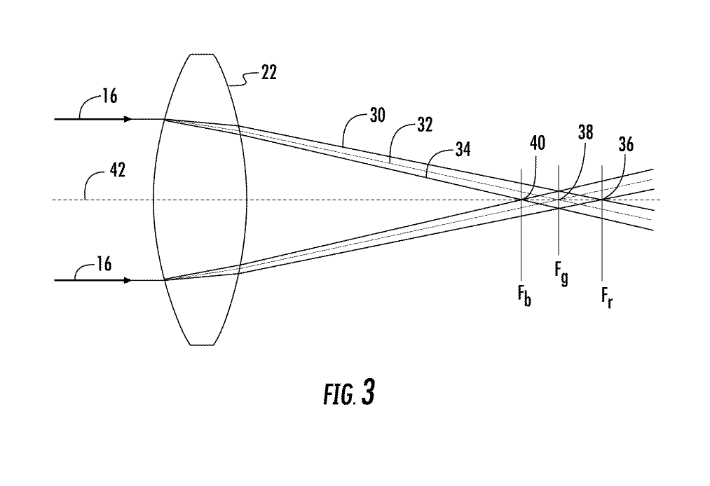

Referring now to FIG. 3, lens 22 acting on light 16 is shown according to one embodiment. Lens 22 is a biconvex lens defining optical axis 43. While as shown in FIG. 3 lens 22 includes generally symmetric front and rear surfaces, in practice the front and rear surfaces of lens 22 are non-symmetric and/or variable to enable focusing of the human eye on objects at various distances. Light 16 enters lens 22 and is refracted toward optical axis 43. As shown in FIG. 3, red light 30 converges along optical axis 43 at focal point 36, green light 32 converges along optical axis 43 at focal point 38, and blue light 34 converges along optical axis 43 at focal point 40.

The differences in focal distances between the red, green, and blue light results from the different colors being associated with light rays of different wavelengths, and the cornea and lens having differing refractive properties for each different wavelength. For example, lens 22 has more focusing power for blue light than red light, resulting in a shorter focal distance for blue light than for red light. The resultant condition is referred to as longitudinal chromatic aberration, and results in fringes of color appearing, for example, along boundaries separating dark and light objects. Incident light 16 in FIG. 3 is illustrated as arriving along the lens's optical axis 43. In cases where light 16 arrives at an angle to optical axis 43, focal points 40, 38, and 36 will be displaced laterally or sideways by different amounts, an effect referred to as lateral chromatic aberration.

Similar aberrations may be introduced by corrective eyewear, such as eyeglasses or contact lenses. For example, referring to FIG. 4, optical train 44 is shown according to one embodiment. As used herein, an optical train refers to the aggregate of the optical components of the eye along with corrective eyewear that may be used in connection with the eye. As shown in FIG. 4, optical train 44 includes lens 22 (e.g., from an eye) and corrective lens 46 (e.g., from a corrective pair of eyeglasses or contact lenses, etc.). As shown in FIG. 4, optical train 44 may produce a chromatic aberration along optical axis 42, as shown by the different focal lengths of focal points 36, 38, 40.

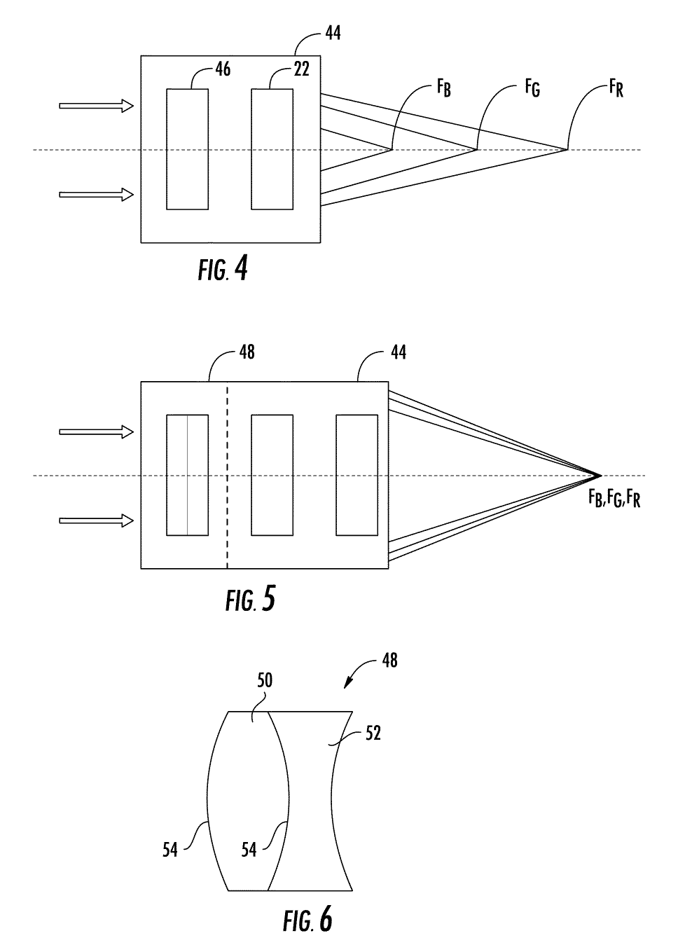

Referring to FIG. 5, supplemental lens assembly 48 is in some embodiments used in combination with optical train 44. Assembly 48 is configured to correct (i.e., to substantially reduce or to eliminate) the chromatic aberration introduced by one or both of eye lens 22 and corrective eyewear lens 46 (e.g., a primary lens or lens assembly, etc.). In one embodiment, corrective lens 46 is configured to correct a user's vision (e.g., near-sightedness, far-sightedness, etc.). However, corrective lens 46 may also introduce chromatic aberration in addition to that naturally existing in a user's eye. As such, supplemental lens assembly 48 is configured to correct the chromatic aberration of the eye and corrective eyewear while having substantially no effect on the net focusing power of the optical train (without lens assembly 48). In some embodiments, lens assembly 48 corrects the chromatic aberration of both an eye and a primary corrective lens. In other embodiments, lens assembly 48 corrects the chromatic aberration of only the eye (e.g., in the case of a user who does not normally wear corrective eyewear but still experiences chromatic aberration naturally occurring within the human eye) or of only the primary corrective lens.

Referring now to FIG. 6, supplemental lens assembly 48 is shown according to one embodiment. As shown in FIG. 6, lens assembly 48 may be a compound or multi-lens assembly including a number of individual lenses coupled together. In one embodiment, lens assembly 48 includes lens 50 (e.g., a first lens or component) and lens 52 (e.g., a second lens or component). Lenses 50, 52 are configured to correct chromatic aberration introduced by an eye and/or corrective eyewear while having substantially no net focusing power. Lens assembly 48 may be implemented in a variety of types of eyewear, including contact lenses, eyeglasses, thin films or layers applied to eyeglasses, add-on lenses intended to be coupled to eyeglasses (e.g., via clips, etc.), and the like.

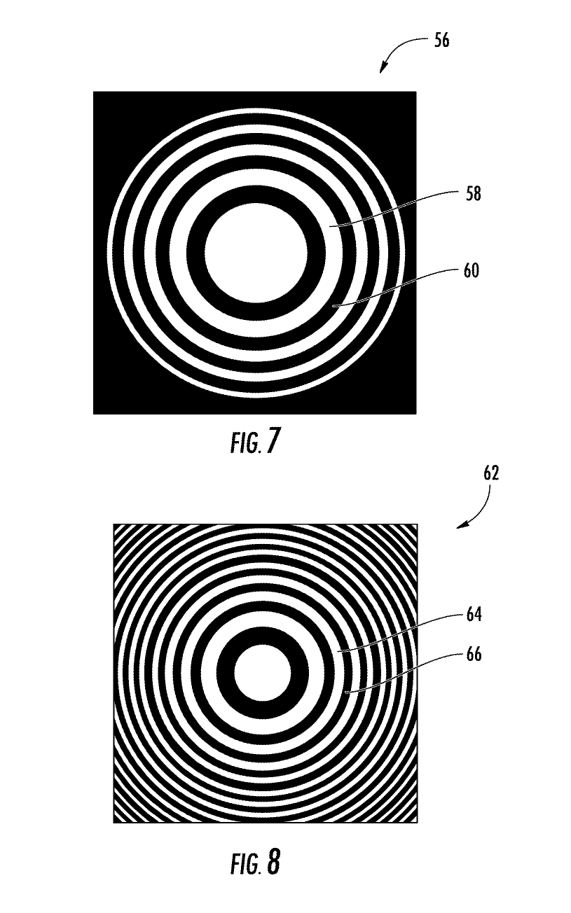

According to one embodiment, lens 50 is a diffractive lens and has a positive optical power, and lens 52 is a refractive lens and has a negative optical power. In one embodiment, the diffractive lens includes a diffractive lens profile (e.g., a diffractive pattern or grating, etc.) configured to diffract light passing through the lens. For example, referring to FIGS. 7-8, schematic representations of diffractive lens profiles are shown according to alternative embodiments. As shown in FIGS. 7-8, diffractive lens profiles may include a series of radially symmetric rings, or grooves, that act to vary the transmissivity of the light or to vary the phase of the light for various wavelengths of light based on the characteristics of the rings (e.g., the width of the rings, the spacing of the rings, the depth of the rings, the slopes of the surfaces forming the rings, and the like).

Generally, diffractive lens profiles can be described by a two-dimensional thickness profile which varies along the surface in which the profile is embedded. The diffractive lens profile is characterized by both the curves defining the grooves (e.g., circular rings, straight lines, etc.), as well as the thickness profile within each groove. The grooves can take various thickness profile shapes, including square/binary, sinusoidal, sawtooth, and the like. For example, while FIG. 7 shows a generally binary diffractive lens profile 56 having rings 58, 60, FIG. 8 shows a sinusoidal diffractive lens profile 62 having rings 64, 66. In a binary profile, thickness generally changes abruptly between 0% and 50% (or, alternatively, may involve N steps rather than just two, for instance 4 steps with thicknesses of 0, 25, 50, and 75%), while in blazed profiles (e.g., a sinusoidal profile or a sawtooth profile) thickness typically changes more gradually between 0% and 100%. In a sinusoidal profile, the grooves, or rings, are formed by the sinusoidal contour extending radially outward from the center of the diffraction pattern.

Diffractive lenses inherently have a strong chromatic variation in their optical power, i.e., optical power for a diffractive lens is proportional to wavelength. Relative to the optical power at a reference wavelength λ*:

Φ(λ)=Φ*λ/λ*=Φ*[1+u]

The chromatic behavior of a diffractive lens's optical power is both stronger (α=1) and opposite in sign that of conventional lenses.

Use of a diffractive lens profile alone is sufficient to correct chromatic aberrations in the eye or in a primary corrective lens, but addition of chromatic optical power Φ* u will be accompanied by an achromatic shift Φ* in the resultant optical power of the system. By combining both a refractive and a diffractive correction, it is possible to correct both chromatic and achromatic optical powers (i.e., to achieve a desired achromatic target optical power as well as a desired chromatic correction). It should also be noted, that if the patient wants a non-zero chromatic variation in their target optical power, this can also be achieved by the appropriate choices of the diffractive and refractive corrections. In an embodiment, a user is already satisfied with his eye's achromatic optical power (either that of the eye itself, or the combined optical power of his eye in conjunction with an existing primary corrective lens), but desires a specified amount of chromatic correction. In such cases, supplemental lens assembly 48 can be designed to provide the user with the specified amount of chromatic optical power, but with no achromatic optical power. Supplemental lens assembly 48 comprises a diffractive lens having achromatic optical power Φ_{D} in series with a negative refractive lens (e.g., the diffractive lens is applied to a surface of the negative refractive lens) having achromatic optical power Φ_{R}=−Φ_{D}. In such an embodiment, supplemental lens assembly 48 provides only chromatic optical power of size Φ(u)=(1+α)Φ_{D}u. Matching this to the desired amount of chromatic correction Φ_{c}u determines the amount of diffractive power needed in supplemental lens assembly 48, i.e., Φ_{D}=Φ_{c}/(1+α).

Referring further to FIGS. 7-8, by controlling the characteristics of the diffractive lens profile applied to a supplemental lens, the corresponding changes to the focusing power of an optical train can be controlled. For example, the diffractive lens profile may be configured so as to minimize chromatic optical power or aberrations in certain wavelengths. In some embodiments, the diffractive lens profile is applied to so as to minimize chromatic optical power or aberrations near a single wavelength (e.g., red, green, blue, etc.) or over a range of wavelengths (red to blue, etc.). The diffractive lens profile may be applied to have a particular spacing profile between rings, a particular depth of groove, a particular slope of a sinusoidal pattern forming the grooves, and so on, in order to provide a desired correction of chromatic aberration.

In some embodiments, the diffractive lens profile is applied to substantially all of a supplemental lens assembly through which visible light passes. In other embodiments, the diffractive lens profile is applied to only a portion of the area through which visible light passes. According to various alternative embodiments, in addition to applying or omitting diffractive lens profiles in various portions of the supplemental lens assembly, the diffractive lens profile may be varied between portions of the lens or between lenses of a pair of lenses (e.g., between the left and right lenses of a pair of eyeglasses, contact lenses, etc.).

Referring further to FIG. 6, in some embodiments, rather than or in addition to a diffractive lens, lens assembly 48 includes multiple refractive lenses. For example, in one embodiment, lens assembly 48 includes refractive lenses 50, 52. In one embodiment, lens 50 is a first refractive lens having a positive optical power and lens 52 is a second refractive lens having a negative optical power. In one embodiment, lens 50 has a first dispersion, α₁, and lens 52 has a second dispersion, α₂, greater in magnitude from the first dispersion.

Φ(u)=Φ₁[1−α₁u]+Φ₂[1−α₂u]=(α₂−α₁)Φ₁u, by using Φ₂=−Φ₁

The difference in magnitude between the first and second dispersions of lenses 50, 52 can be selected to correct chromatic aberration (i.e., Φ₁=Φ_{c}/(α₂−α₁). The chromatic aberration can correspond to that of a person's eye, or alternatively, to that resulting from a person's eye in combination with primary corrective eyewear.

As noted above, the various lenses of lens assembly 48 can be configured such that lens assembly 48 has substantially no net achromatic optical power (e.g., at a reference wavelength corresponding to, for example, a visible wavelength such as blue, green, or red; over a range of wavelengths including, for example, visible wavelengths such as blue, green, and/or red, etc.). While certain combinations of lenses making up lens assembly 48 are described herein for purposed of explanation, it should be understood that other combinations of lenses may be used according to various alternative embodiments.

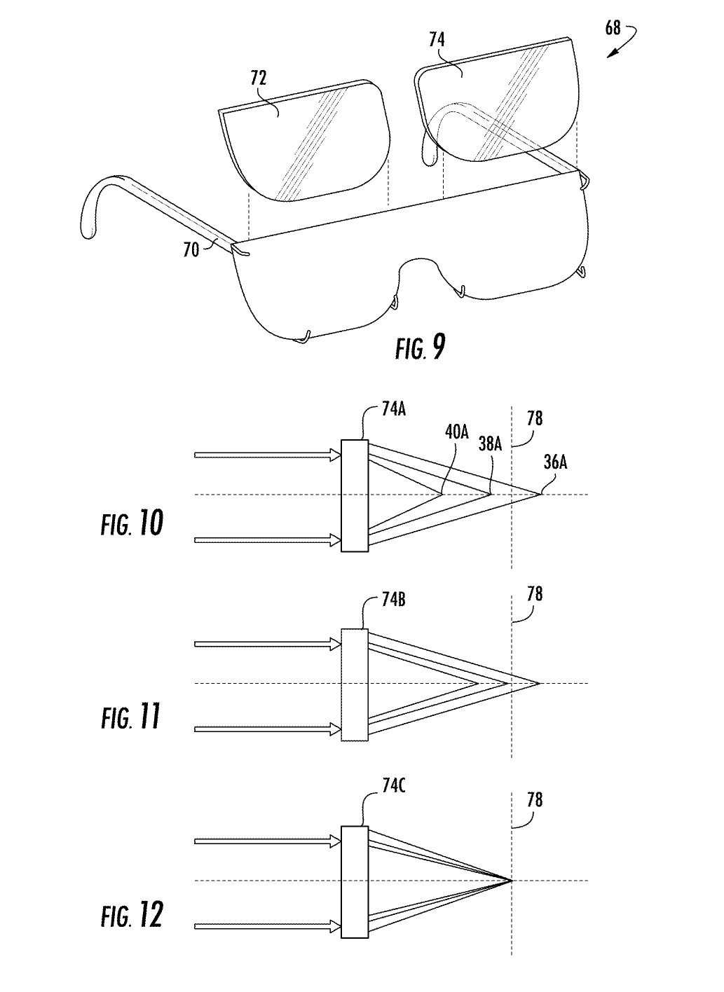

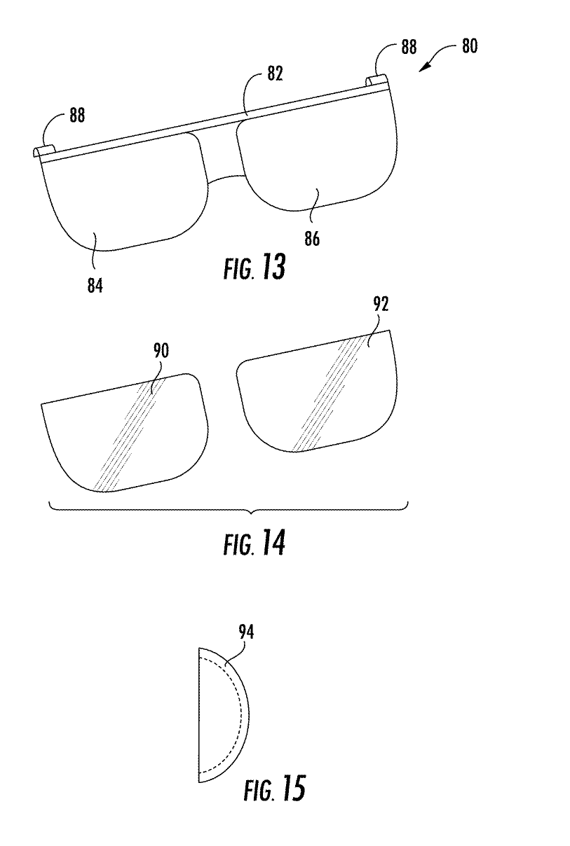

Referring now to FIG. 9, in one embodiment, an evaluation system such as evaluation system 68 may be used to evaluate the degree of chromatic aberration experienced by a patient. System 68 includes frame 70 and sample lenses 72, 74. System 68 is usable to evaluate a degree of chromatic aberration in a patient by facilitating the determination of an appropriate corrective lens assembly. For example, referring to FIGS. 10-12, for a particular patient, light passing through sample lens 74A may have focal lengths 36A, 38A, and 40A (corresponding to, e.g., red, green, and blue colors). Light passing through sample lens 74B may have focal lengths 36B, 38B, and 40B, and light passing through sample lens 74C may have focal lengths 36C, 38C, and 40C. The various focal lengths shown in FIGS. 10-12 represent those experienced by a patient while wearing frame 70 along with one of sample lenses 74A, 74B, 74C. As shown by FIGS. 10-12, sample lens 74C provides the most improvement in chromatic aberration while having no net effect on the focusing power of the optical train (e.g., no net shift of the achromatic focal point 78 of the bare eye or the bare eye in combination with primary corrective eyewear).

Based on use of evaluation system 68, an appropriate supplemental lens assembly can be provided to a patient. It should be noted that the chromatic correction required, and therefore the supplemental lens provided, may vary between eyes (e.g., between the left and right eyes of a user). Furthermore, evaluations may be periodically performed such that the chromatic aberration correction provided by the supplemental lens assembly may be updated accordingly should a person's vision deteriorate with age or other factors.

Referring now to FIGS. 13-15, supplemental lenses may be implemented in a wide variety of eyewear according to various alternative embodiments. For example, as shown in FIG. 13, supplemental lens assembly 80 is configured to be coupled to primary corrective eyewear (e.g., primary eyeglasses, etc.). Supplemental lens assembly 80 in one embodiment includes frame 82, supplemental lenses 84, 86, and fasteners 88. As noted above, lenses 84, 86 are configured to correct the optical aberration of the optical train of a user (e.g., the bare eye in combination with corrective eyewear), and may be coupled to corrective eyewear using fasteners 88 (e.g., clips, etc.). In some embodiments, supplemental lens assembly 80 is configured as conventional eyeglasses (i.e., to attach to the ears of the user), so as to be worn over or under existing primary eyeglasses, or in place of primary eyeglasses.

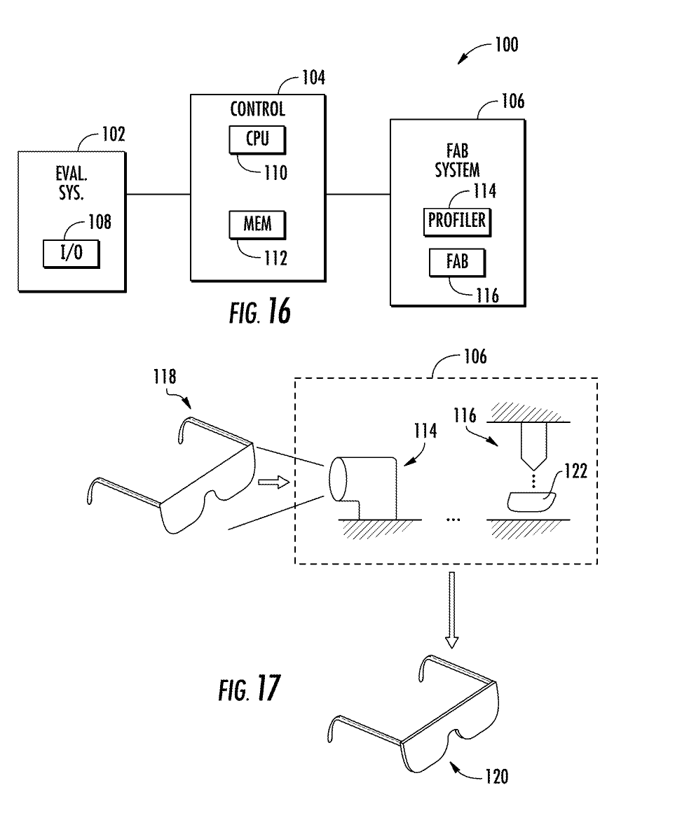

In some embodiments, as shown in FIG. 14, supplemental lenses 90, 92 are configured to couple directly to a surface of one or more primary lenses. Lenses 90, 92 may couple to an inner or an outer surface of primary lenses, and may be provided as a flexible lens. In one embodiment, lenses 90, 92 are configured to be adhered to one or more primary lenses. Referring to FIG. 15, in further embodiments, supplemental lens 94 is a contact lens configured to be worn on an eye of a user. Alternatively, supplemental lens 94 may be an intraocular lens configured for implantation within the eye. Supplemental lenses may be implemented in different ways according to various other alternative embodiments.

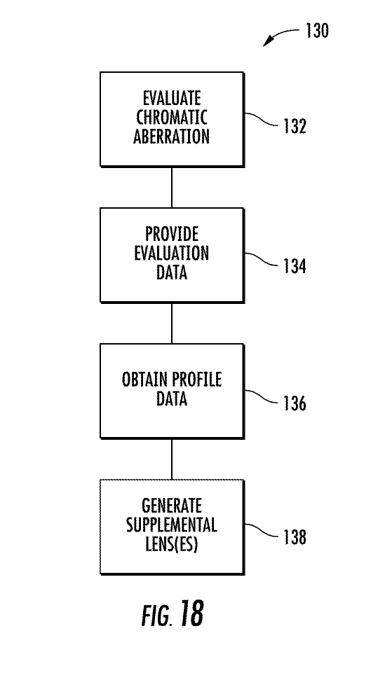

Referring now to FIGS. 16-17, system 100 for evaluating chromatic aberration and fabricating supplemental lenses is shown according to one embodiment. System 100 includes evaluation system 102, control system 104, and fabrication system 106. System 100 may be provided as a physically integrated system, or alternatively, as multiple physically distinct components operatively coupled together. Evaluation system 102 may utilize components similar to those described with respect to FIG. 10 to determine an appropriate supplemental lens configuration for each eye of a user, and input/output device 108 may receive an input identifying the appropriate correction and/or a desired supplemental lens.

Evaluation system 102 is configured to provide evaluation data regarding a patient to control system 104. The evaluation data may be based on an input received from a person (e.g., optometrist or patient, etc.) during an eye exam by way of device 108. Alternatively, evaluation system 102 may include various diagnostic equipment configured to generate evaluation data based on evaluating a patient eye and/or primary corrective eyewear of the For example, evaluation system 102 may be or include other diagnostic devices configured to evaluate one or more portions of the eye, including the cornea, lens, and the like. As noted above, system 102 may also be configured to receive inputs from a user (e.g., an ophthalmologist, etc.) regarding a visual analysis and/or examination of an eye. Such examinations can use instruments such as phoropters (for classical focal quality analysis), aberrometers (for wavefront analysis), or the like. These examinations can be performed for a single wavelength, for a simultaneous suite of wavelengths (e.g., white light), or sequentially using different colors (thereby measuring chromatic aberrations). Evaluation system 102 forwards evaluation data to control system 104 (e.g., a controller, processing circuit, etc.).

Control system 104 includes processor 110 and memory 112. Processor 104 may be implemented as a general-purpose processor, an application specific integrated circuit (ASIC), one or more field programmable gate arrays (FPGAs), a digital-signal-processor (DSP), a group of processing components, or other suitable electronic processing components. Memory 106 is one or more devices (e.g., RAM, ROM, Flash Memory, hard disk storage, etc.) for storing data and/or computer code for facilitating the various processes described herein. Memory 106 may be or include non-transient volatile memory or non-volatile memory. Memory 106 may include database components, object code components, script components, or any other type of information structure for supporting the various activities and information structures described herein. Memory 106 may be communicably connected to processor 104 and provide computer code or instructions to processor 104 for executing the processes described herein.

Control system 104 is configured to control operation of fabrication system 106 and/or provide evaluation data to fabrication system 106. For example, control system 102 may instruct profiler 114 to obtain profile data for primary eyewear 118 (e.g., by way of an imaging or profiling device, etc.). Based on the profile data and the evaluation data, a fabrication device 116 (e.g., a 3-D printer, a laser device, etc.) can select one or more appropriate lens templates 122 and form the required supplemental lens assembly. Fabrication device 116 may be configured to match an outer periphery of existing eyewear, match a curvature of existing eyewear, match a lens color of existing eyewear, etc. when fabricating supplemental lenses. In one embodiment, fabrication device 116 is further configured to apply or couple one or more supplemental lenses to primary eyewear to form supplemental eyewear 120. The supplemental lenses may be applied directly to one or more primary lenses (e.g., as described with respect to FIG. 14), or alternatively, may be provided as a supplemental lens assembly configured for removable attachment to primary eyewear (e.g., as described with respect to FIG. 13).

Referring now to FIG. 18, method 130 of providing one or more supplemental lens assemblies is shown according to one embodiment. A degree of chromatic aberration is evaluated (132). In some embodiments, evaluating chromatic aberration is done using an evaluation system such as those described herein to generate evaluation data indicative of the degree of chromatic aberration correction required. The evaluation may be performed based on a bare eye, or an eye in combination with primary corrective eyewear. Further, each eye of a person may be evaluated independently. The evaluation data is provided (134). In some embodiments, evaluation data is provided by an evaluation system to a fabrication system, such as fabrication system 106. Profile data is obtained (136). Profile data may include data regarding a person's eye, or alternatively, data regarding primary corrective eyewear of a person, such that one or more supplemental lenses can be accommodated by the eye/eyewear. Based on the evaluation data and the profile data, supplemental lenses are generated (138). In one embodiment, one or more supplemental lenses are generated from lens templates selected based on the evaluation and/or profile data. The supplemental lenses may be coupled to one or more primary lenses, or alternatively, provided as a physically distinct component (e.g., a supplemental eyewear assembly, a contact lens, etc.) configured to supplement the person's optical train.

In an embodiment, a method of providing a supplemental lens assembly to a user comprises presenting to the user a plurality of supplemental lens assemblies, each supplemental lens assembly having a different amount of chromatic optical power, and hence capable of providing the user with a different amount of chromatic correction. The user is able to temporarily try out one or more of the supplemental lens assemblies, so as to select a particular supplemental lens assembly preferable to him. In some embodiments, each of the supplemental lens assemblies provides positive chromatic optical power (i.e., focuses red light more strongly than blue light). In some embodiments, each of the supplemental lens assemblies comprises an indicia identifying the amount of chromatic optical power provided by it. These indicia may comprise an absolute value (e.g., a diopter difference in focal power between red and blue light) or a relative value (e.g., large, medium, or small chromatic power), and may comprise text, numbers, symbols, colors, or the like. In some embodiments, the supplemental lens assemblies have substantially zero achromatic optical power, while in other embodiments one or more of the supplemental lens assemblies may provide both a specified achromatic optical power as well as a specified chromatic optical power.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A wearable lens assembly, comprising:
a first lens having a first optical power; and
a second lens coupled to the first lens and having a second optical power substantially equal in magnitude and opposite in power to the first optical power;
wherein the first and second lenses are collectively configured to correct chromatic aberration in at least one of an eye of a user and corrective eyewear of a user.

2. (canceled)

3. The assembly of claim 1, wherein the first lens is a diffractive lens including a diffractive profile and having positive optical power, and wherein the second lens is a refractive lens having a negative optical power.

4. The assembly of claim 3, wherein the diffractive profile is applied to only a portion of a surface of the first lens.

5. (canceled)

6. The assembly of claim 1, wherein the first lens is a refractive lens having a positive optical power and a first dispersion, and wherein the second lens is a refractive lens having a negative optical power and a second dispersion greater than the first dispersion.

7. The assembly of claim 6, wherein a difference between the first dispersion and the second dispersion is configured to correct the chromatic aberration.

8. The assembly of claim 1, wherein the first and second lenses are collectively configured to correct chromatic aberration in the eye without corrective eyewear.

9-13. (canceled)

14. The assembly of claim 1, wherein the first and second lenses form a contact lens configured to be worn on the eye.

15. The assembly of claim 1, wherein the first and second lenses form a removable supplemental lens for the corrective eyewear.

16. The assembly of claim 15, wherein the removable supplemental lens is coupled to a primary lens.

17. The assembly of claim 15, wherein the removable supplemental lens is coupled to a surface of the primary lens.

18. The assembly of claim 17, wherein the surface is an inner surface of the primary lens.

19. The assembly of claim 17, wherein the surface is an outer surface of the primary lens.

20. (canceled)

21. The assembly of claim 1, wherein the first and second lenses form an intraocular lens.

22-25. (canceled)

26. A supplemental eyeglass assembly configured to supplement primary corrective eyewear, comprising:
a first lens assembly configured to correct a first chromatic aberration resulting from a first eye of a user and a first primary lens of the primary corrective eyewear;
a second lens assembly configured to correct a second chromatic aberration resulting from a second eye of the user and a second primary lens of the primary corrective eyewear;
wherein the first and second lens assemblies are configured to be coupled to the primary corrective eyewear and have substantially no effect on the achromatic focusing power of the primary corrective eyewear.

27. The assembly of claim 26, wherein at least one of the first lens assembly and the second lens assembly includes a first lens coupled to a second lens, wherein the first lens is a diffractive lens including a diffractive profile and having positive optical power, and wherein the second lens is a refractive lens having a negative optical power.

28. The assembly of claim 27, wherein the diffractive profile is applied to only a portion of a surface of the first lens.

29. (canceled)

30. The assembly of claim 26, wherein at least one of the first lens assembly and the second lens assembly includes a first lens coupled to a second lens, wherein the first lens is a refractive lens having a positive optical power and a first dispersion, and wherein the second lens is a refractive lens having a negative optical power and a second dispersion greater than the first dispersion.

31. The assembly of claim 30, wherein a difference between the first dispersion and the second dispersion is configured to correct at least one of the first chromatic aberration and the second chromatic aberration.

32. The assembly of claim 26, wherein at least one of the first chromatic aberration and the second chromatic aberration includes a difference in focal lengths between at least two different colors.

33-35. (canceled)

36. The assembly of claim 26, wherein the first and second lens assemblies form a removable supplemental lens assembly for the corrective eyewear.

37. The assembly of claim 36, wherein the removable supplemental lens assembly is coupled to a surface of the primary lens.

38. The assembly of claim 37, wherein the surface is an inner surface of the primary lens.

39. The assembly of claim 37, wherein the surface is an outer surface of the primary lens.

40. The assembly of claim 36, wherein the removable supplemental lens assembly is configured to be coupled to a frame of the corrective eyewear.

41-44. (canceled)

45. A corrective eyeglass assembly, comprising:
a primary wearable lens assembly configured to provide a focal shift to light incident upon a user eye at a reference wavelength; and
a secondary wearable lens assembly separable from the primary wearable lens assembly and configured to correct a chromatic aberration resulting from at least one of the user eye and the primary wearable lens assembly;
wherein the secondary wearable lens assembly has substantially no effect on the focal shift provided by the primary wearable lens assembly at the reference wavelength.

46-48. (canceled)

49. The assembly of claim 45, wherein the secondary wearable lens assembly includes a first lens and a second lens, wherein the first lens is a diffractive lens including a diffractive profile and having positive optical power, and wherein the second lens is a refractive lens having a negative optical power.

50. The assembly of claim 49, wherein the diffractive profile is applied to only a portion of a surface of the first lens.

51. The assembly of claim 49, wherein the diffractive profile defines at least one of concentric circles, concentric ellipses, and straight parallel lines.

52. The assembly of claim 45, wherein the secondary wearable lens assembly includes a first lens and a second lens, wherein the first lens is a refractive lens having a positive optical power and a first dispersion, and wherein the second lens is a refractive lens having a negative optical power and a second dispersion greater than the first dispersion.

53. The assembly of claim 52, wherein a difference between the first dispersion and the second dispersion is configured to correct the chromatic aberration in the eye of the user and the primary wearable lens assembly.

54-57. (canceled)

58. The assembly of claim 45, wherein the secondary wearable lens assembly includes a contact lens configured to be worn on the eye.

59. The assembly of claim 45, wherein the secondary wearable lens assembly includes a removable supplemental lens for the primary wearable lens assembly.

60. The assembly of claim 59, wherein the removable supplemental lens is coupled to the primary wearable lens assembly.

61. The assembly of claim 59, wherein the removable supplemental lens is coupled to a lens surface of the primary wearable lens assembly.

62-64. (canceled)

65. The assembly of claim 45, wherein the secondary wearable lens assembly includes an intraocular lens.

66-108. (canceled)
